(19) United States
(12) Patent Application Publication
(10) Pub. No.: US 2022/0184338 A1
(43) Pub. Date: Jun. 16, 2022

(54) PORTABLE HUMIDIFICATION APPARATUS FOR TRACHEOSTOMY PATIENTS

(71) Applicant: Henry Ford Health System, Detroit, MI (US)

(72) Inventors: Khaled KASHLAN, Suwanee, GA (US); Justinn EDDIE, Cincinnati, OH (US)

(21) Appl. No.: 17/547,346

(22) Filed: Dec. 10, 2021

Related U.S. Application Data
(60) Provisional application No. 63/123,620, filed on Dec. 10, 2020.

(51) Int. Cl. A61M 16/16; A61M 16/04; A61M 16/10

(52) U.S. Cl. CPC A61M 16/16; A61M 2205/502; A61M 16/1075; A61M 16/0465

(57) ABSTRACT

A portable humidification apparatus for a patient having a tracheostomy including a tracheostomy tube includes an adapter having a central lumen and arranged to be coupled to the tracheostomy tube, the adapter including a first magnet disposed therein. A vaporizer assembly is configured to be removably coupled to the adapter in fluid communication with the central lumen and includes a second magnet disposed therein. The first magnet and the second magnet releasably connect the adapter and the vaporizer assembly and allow for relative rotation between the adapter and the vaporizer assembly when connected. An ultrasonic emitter is disposed within the vaporizer assembly for generating a water mist from water in a water supply system in fluid communication with the vaporizer assembly, wherein the water mist is ducted from the vaporizer assembly to the central lumen of the adapter to provide humidification of air inspired by the patient through the tracheostomy tube.

### CROSS-REFERENCE TO RELATED APPLICATIONS

This application claims the benefit of U.S. provisional application Ser. No. 63/123,620 filed Dec. 10, 2020, the disclosure of which is hereby incorporated in its entirety by reference herein.

### TECHNICAL FIELD

Embodiments relate to a portable humidification apparatus, such as for tracheostomy patients.

### BACKGROUND

Humidification of air is important for proper functioning of the lung. If the air entering into the lungs is dry, then mucus in the respiratory system will harden and remain stuck in the airway, possibly to a point where breathing becomes compromised. If the cilia in the trachea are in an unfavorably dry environment, mucociliary clearance becomes impaired and unwanted debris introduced into the lungs will remain in the lungs.

Thankfully, the human body has a natural means to humidify air. When air is inhaled through the nose, it picks up moisture and heat from the turbinates, which are horizontally-oriented structures in the nasal cavity that produce mucus and which clean and humidify air. The humidification process continues as the air passes down the airway toward the lungs.

When a person is unable to breathe on their own, a tracheostomy may be indicated. A tracheostomy is an opening surgically created through the neck into the trachea to allow direct access to the airway, bypassing the nose and mouth. A tracheostomy tube is placed through this opening to allow for inspiration/expiration and also to remove secretions from the lungs. Because this procedure bypasses the nose and upper airway with air entering directly into the trachea, the body's natural respiratory system is no longer able to adequately humidify inhaled gases. Without warmth and moisture, the person is at risk of additional breathing problems.

Currently, the most common method for artificial humidification in tracheostomy patients is a humidification-moisture exchanger (HME). This is a sponge that is placed at the proximal end of the tracheostomy tube that traps the expired humidified air and then uses this trapped humidity to add moisture to the dry inspired air. However, HMEs are not completely effective in trapping all the water from the expired air and, if a patient coughs, the mucus expelled will completely occlude the HME and require the user to replace it. Patients may also find that HMEs restrict their breathing.

### SUMMARY

In one or more embodiments, a portable humidification apparatus for a patient having a tracheostomy including a tracheostomy tube is provided. The apparatus includes an adapter having a central lumen and arranged to be coupled to the tracheostomy tube, the adapter including a first magnet disposed therein. A vaporizer assembly is configured to be removably coupled to the adapter in fluid communication with the central lumen and includes a second magnet disposed therein. The first magnet and the second magnet releasably connect the adapter and the vaporizer assembly and allow for relative rotation between the adapter and the vaporizer assembly when connected. An ultrasonic emitter is disposed within the vaporizer assembly for generating a water mist from water in a water supply system in fluid communication with the vaporizer assembly, wherein the water mist is ducted from the vaporizer assembly to the central lumen of the adapter to provide humidification of air inspired by the patient through the tracheostomy tube.

In one or more embodiments, the adapter includes a main portion having a first end and a second end, and a branch portion extending from the main portion, wherein the central lumen is disposed in the main portion and the branch portion is in fluid communication with the central lumen, wherein the first end is arranged to be coupled to the tracheostomy tube and the second end is open to an external atmosphere. The first magnet may be disposed within the branch portion.

In one or more embodiments, the vaporizer assembly includes a vaporizer housing and a cover received on the vaporizer housing, wherein the cover includes a central port through which the water mist can be expelled, the central port sized to be received by the adapter to transfer the water mist into the central lumen. The vaporizer assembly may include a seal arranged to receive the ultrasonic emitter. A particulate filter may be removably disposed within the central lumen.

In one or more embodiments, a printed circuit board (PCB) including a microcontroller unit and a power supply may be provided in electrical communication with the ultrasonic emitter. A user interface may be provided in electrical communication with the PCB, wherein the user interface is configured to allow selection of at least one of a power state, a humidity level, and a temperature of the water mist. A heating element may be provided in electrical communication with the PCB and disposed in the vaporizer assembly downstream from the ultrasonic emitter to heat the water mist generated by the ultrasonic emitter.

In one or more embodiments, a portable humidification apparatus for a patient having a tracheostomy including a tracheostomy tube is provided. The apparatus includes an adapter including a main portion having a central lumen, the main portion having a first end arranged to be coupled to the tracheostomy tube and a second end open to an external atmosphere, and a branch portion extending from the main portion and in fluid communication with the central lumen, the adapter including a first magnet disposed in the branch portion. A vaporizer assembly is configured to be removably coupled to the adapter in fluid communication with the central lumen and includes a second magnet disposed therein. The first magnet and the second magnet releasably connect the adapter and the vaporizer assembly and allow for relative rotation between the adapter and the vaporizer assembly when connected. A water supply system is provided in fluid communication with the vaporizer assembly, and an ultrasonic emitter is disposed within the vaporizer assembly for generating a water mist from water in the water supply system, wherein the water mist is ducted from the vaporizer assembly to the central lumen of the adapter to provide humidification of air inspired by the patient through the tracheostomy tube.

In one or more embodiments, the water supply system includes a water reservoir configured to contain water, a connector tube coupled to the water reservoir, and a wick disposed within the connector tube and in fluid communication with the water reservoir, wherein the wick is arranged to transmit water from the water reservoir to the ultrasonic emitter. The vaporizer assembly may include a hollow stem arranged to be coupled to the connector tube, the stem including a lumen which is arranged to receive the wick.

In one or more embodiments, a portable humidification apparatus for a patient having a tracheostomy including a tracheostomy tube is provided. The apparatus includes an adapter including a main portion having a central lumen, the main portion having a first end arranged to connect to the tracheostomy tube and a second end open to an external atmosphere, and a branch portion extending from the main portion and in fluid communication with the central lumen, the adapter including a first magnet disposed therein. A vaporizer assembly is removably coupled to the adapter to be in fluid communication with the central lumen and including a second magnet disposed therein. The first magnet and the second magnet releasably connect the adapter and the vaporizer assembly and allow for relative rotation between the adapter and the vaporizer assembly when connected. A water supply system is in fluid communication with the vaporizer assembly, the water supply system including a water reservoir configured to contain water. An ultrasonic emitter is disposed within the vaporizer assembly for generating a water mist from water in the water reservoir, wherein the water mist is arranged to flow from the vaporizer assembly to the central lumen of the adapter to provide humidification of air inspired by the patient through the tracheostomy tube. The apparatus further includes a storage container arranged to be worn or carried by the patient and housing the water reservoir.

In one or more embodiments, a printed circuit board (PCB) including a microcontroller unit and a power supply may be provided in electrical communication with the ultrasonic emitter, and a user interface may be provided in electrical communication with the PCB, wherein the user interface is configured to allow selection of at least one of a power state, a humidity level, and a temperature of the water mist. The storage container may include a remote housing for supporting the PCB and the user interface.

### BRIEF DESCRIPTION OF THE DRAWINGS

FIG. 1 is an illustration of a portable humidification apparatus according to one or more embodiments connected to a tracheostomy tube and shown in position with respect to a patient's neck;

FIG. 2 is a perspective view of the portable humidification apparatus connected to a tracheostomy tube;

FIG. 3 is a perspective view of an adapter of the portable humidification apparatus disconnected from a vaporizer assembly of the apparatus;

FIG. 4 is a perspective view of the adapter disconnected from the vaporizer assembly;

FIG. 5 illustrates cross-sectional views of the adapter and the vaporizer assembly;

FIG. 6 is a perspective view of the adapter;

FIG. 7 is a top perspective view of vaporizer housing cover according to one or more embodiments;

FIG. 8 is a bottom perspective view of a cover for the vaporizer housing;

FIG. 9 is a perspective view of a first seal within the vaporizer housing;

FIG. 10 is a cross-sectional view of the first seal of FIG. 9;

FIG. 11 is a perspective view of an ultrasonic emitter according to one or more embodiments;

FIG. 12 is an illustration of the ultrasonic emitter in electrical communication with a printed circuit board;

FIG. 13 is a perspective view of the vaporizer housing;

FIG. 14 is a cross-sectional view of the vaporizer housing of FIG. 13;

FIG. 15 is an illustration of a wick inside a connector tube according to one or more embodiments;

FIG. 16 is an illustration showing the portable humidification apparatus connected a water reservoir which may be disposed in a backpack;

FIG. 17 is an illustration depicting a user interface which may be connected to the backpack according to one or more embodiments; and

FIG. 18 is a photograph illustrating placement of the portable humidification apparatus in position with respect to a patient's neck while wearing the backpack.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

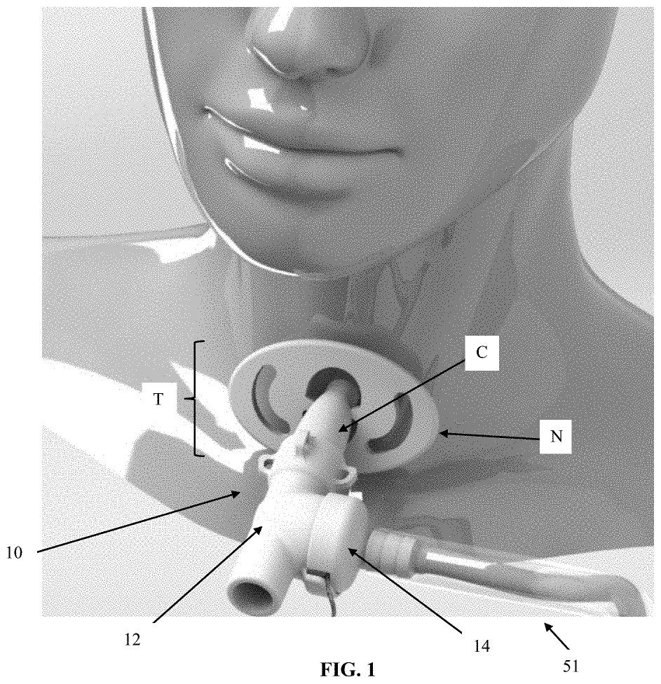

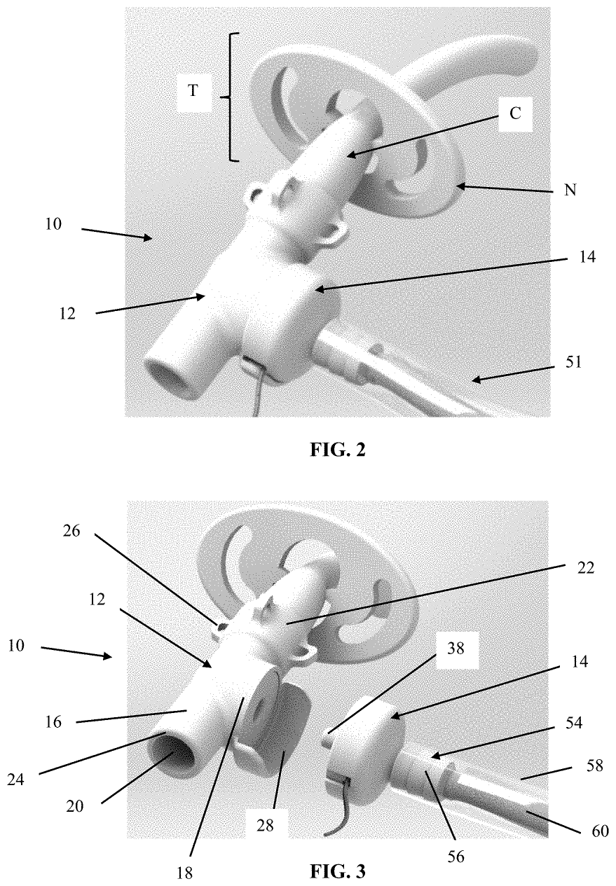

A portable humidification apparatus is disclosed herein that can function as a replacement to the body's natural humidification process for tracheostomy patients. As is known in the art, a tracheostomy tube T includes an outer cannula C and neck plate N as shown in FIGS. 1-3. The portable humidification apparatus 10 includes an adapter 12 arranged to be coupled to the standard end of the tracheostomy tube T (outer cannula C), and a vaporizer assembly 14 configured to be removably coupled to the adapter 12 to provide humidification of the inspired air as described below.

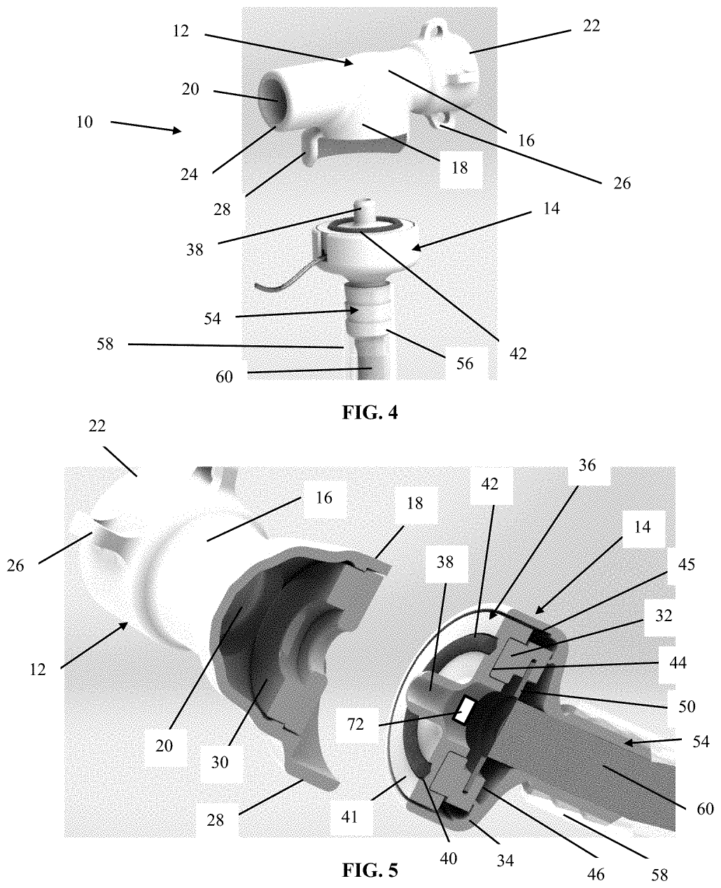

With reference to FIGS. 1-6, in one or more embodiments the adapter 12 may be generally T-shaped, with a hollow main portion 16 and a branch portion 18 extending generally perpendicularly from the main portion 16. The main portion 16 has a central lumen 20 with a first end 22 and a second end 24, wherein the branch portion 18 is in fluid communication with the central lumen 20. The main portion 16 and central lumen 20 may be generally cylindrical as shown, wherein the first end 22 is sized to receive or arranged to be coupled to any tracheostomy tube T, such as via an interference fit, and the second end 24 is open to the external atmosphere. The first and second ends 22, 24 and the central lumen 20 are sized to ensure that no restrictions in airflow occur, allowing for optimal breathing conditions for the patient. One or more hook members 26 may be provided around an exterior circumference of the first end 22 as shown, wherein these hook members 26 provide a secure connection point to any accessory and can provide additional support in the event of more rigorous patient activity. As best shown in FIGS. 3-6, the branch portion 18 may include a lip member 28 which may be sized to receive the vaporizer assembly 14 thereon and serve as a mechanical support for connection of the adapter 12 to the vaporizer assembly 14. In one non-limiting embodiment, the adapter 12 may have a length of approximately 2 in. The adapter 12 may be constructed from a plastic material, and may be washable and reusable.

As shown in the cross-sectional view of FIG. 5, in one or more embodiments a first magnet 30 is disposed within the adapter 12, such as within the branch portion 18, and a second magnet 32 is disposed within the vaporizer assembly 14, where the first and second magnets 30, 32 are utilized to create an attractive force that releasably connects the adapter 12 and the vaporizer assembly 14 together. This non-permanent attraction of the first and second magnets 30, 32 is strong enough to function through the resistance of plastic components within the adapter 12 and the vaporizer assembly 14 and the separation distance of the first and second magnets 30, 32, but not so strong as to prevent disconnection of the adapter 12 and the vaporizer assembly 14 or to damage electrical components of the apparatus 10. The first and second magnets 30, 32 may be encapsulated or coated to maintain effectiveness over the life of the portable humidification apparatus 10.

The first and second magnets 30, 32 may each have an annular or ring geometry as shown, maximizing surface area with a relatively thin profile. In one non-limiting embodiment, the first and second magnets 30, 32 create approximately 1 lb. of pull force, and function as a quick connect/disconnect system for the adapter 12 and the vaporizer assembly 14 which is strong enough to hold these components of the apparatus 10 together, but weak enough to safely disconnect these components on impact or when pulled. The magnetic connection also allows for relative rotation between the adapter 12 and the vaporizer assembly 14 for positioning and to accommodate movement of the patient. As an alternative to magnets, the apparatus 10 can use other means of attachment, such as clips, or buttons.

Referring to FIGS. 3-5 and 7-8, the vaporizer assembly 14 includes a vaporizer housing 34 in which various components are disposed and a cover 36 which is received on the vaporizer housing 34 and serves as the interface with the adapter 12. The cover 36 may be generally disc-shaped and includes a central port 38 extending therefrom through which water mist can be expelled as described below. The central port 38 may be generally cylindrical and may be sized to be received in the branch portion 18 and through the first magnet 30. The central port 38 has a sufficient length to easily transfer the water mist efficiently into the central lumen 20 to mix with ambient air inside the adapter 12 and into the patient's breathing air pathway. With the central port 38 extending into the adapter 12, this configuration provides another structural safeguard to prevent unintended disconnection of the vaporizer assembly 14 and the adapter 12.

In one or more embodiments, an annular groove 40 is formed in a top surface 41 of the cover 36, as best shown in FIGS. 5 and 7. The annular groove 40 is sized to at least partially receive a first seal 42 which prevents liquid from escaping the central port 38 as it condenses. The first seal 42 may be molded into or adhered to the groove 40 and may have a durometer, for example, of 10-20 shore A. The first seal 42 can be constructed from a chemical/temperature resistant rubber such as, but not limited to, VITON™. An annular recess 44 may be formed in a bottom surface 45 of the cover 36 and sized to at least partially receive the second magnet 32 (see FIGS. 5 and 8). The cover 36 may be constructed with sufficient thickness to provide structural stability while allowing for adequate attractive force between the first and second magnets 30, 32.

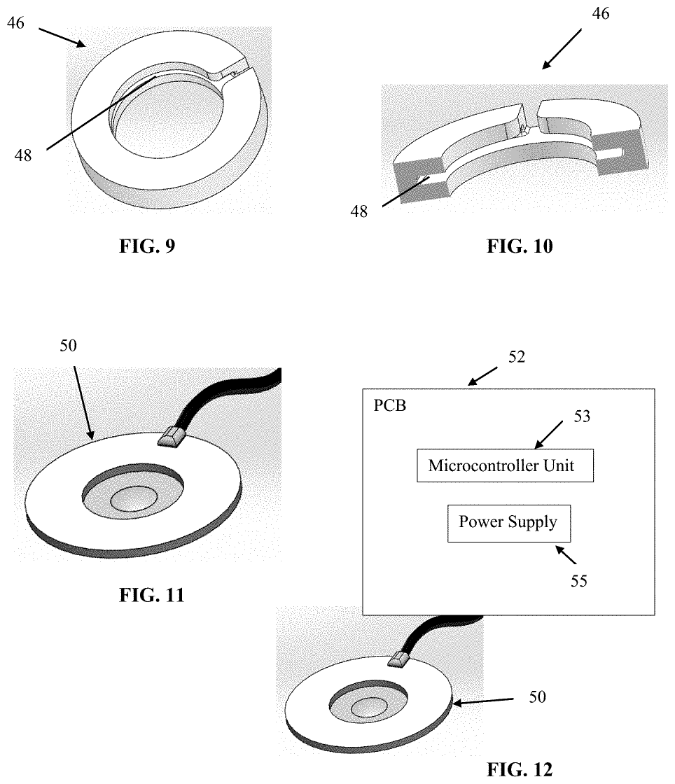

With reference to FIGS. 5 and 9-10, a second seal 46 may be provided below the second magnet 32. In one non-limiting embodiment, the second seal 46 is constructed from a silicone material and may have a durometer of, for example, 20-30 Shore A. The second seal 46 may include an internal groove 48 arranged to receive an ultrasonic emitter 50 which functions to create water vapor or water mist as described further below. The second seal 46 prevents electronics associated with the ultrasonic emitter 50 from being contacted with water. The second seal 46 also creates a protective barrier for the ultrasonic emitter 50, decoupling and preventing the ultrasonic emitter 50 from transmitting vibrations through the apparatus 10 which would shorten its life or cause damage to the apparatus 10. Alternatively, the ultrasonic emitter 50 could be disposed elsewhere within the vaporizer assembly 14. Although an ultrasonic emitter 50 is shown and described herein, it is understood that the apparatus 10 could alternatively use other means of vaporizing water.

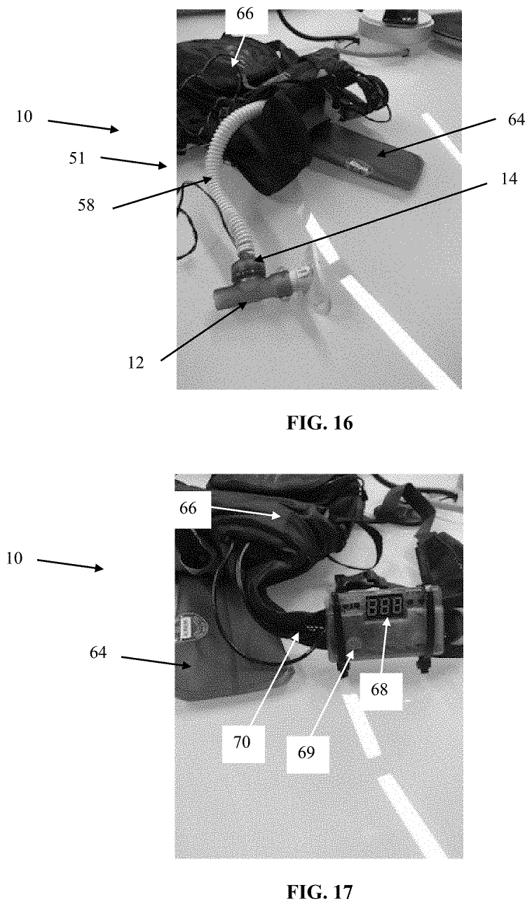

The ultrasonic emitter 50 is best shown in FIGS. 11 and 12. In one non-limiting embodiment, the ultrasonic emitter 50 includes a 0.625 in diameter ceramic ultrasonic disk which generates a resonant frequency at 108 kHz and functions at 5 Volts DC. In this non-limiting embodiment, the ultrasonic emitter 50 may have a resonant impedance of less than 150 ohms, a static capacitance of 3000 pF, and a power driver capable of spraying a volume of 50-100 ml/h. To produce water mist, the ultrasonic emitter 50 is in fluid communication with a water supply system 51 (FIG. 16) which supplies water to an underside of the ultrasonic emitter 50 and the water mist is transferred across a permeable surface in the center of the ultrasonic emitter 50. The water mist is then ducted through the central port 38 of the cover 36 and into the central lumen 20 of the adapter 12 for delivery to the patient with no other mechanical assistance required.

The ultrasonic emitter 50 is in electrical communication with a printed circuit board (PCB) 52 (FIG. 12) which supports electrical components including a microcontroller unit 53 and a power supply 55, such as a rechargeable battery which may be charged via a USB connection or the like. The PCB 52 may include a variable voltage regulator which allows for varying voltage to be supplied to the ultrasonic emitter 50, thus varying the amount of water mist emitted by the ultrasonic emitter 50 and allowing the humidity to be selectable and customized as desired by the user. The PCB 52 may be disposed within the vaporizer housing 34 or external thereto as described further below with reference to FIGS. 16-18.

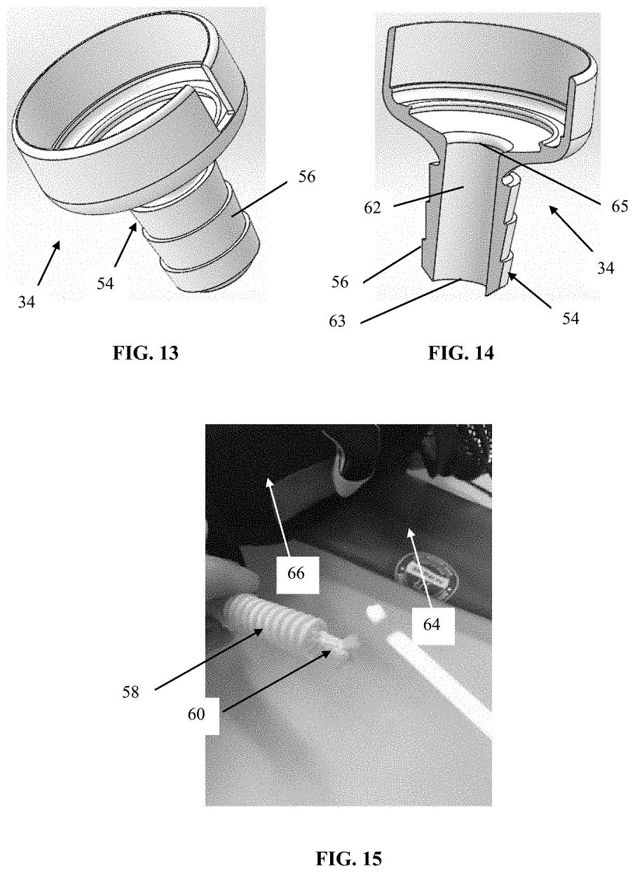

The vaporizer housing 34 serves to support the ultrasonic emitter 50 and also serves as a connector to the water supply system 51 containing water used to create the water mist. In one or more embodiments, the vaporizer housing 34 includes a hollow stem 54 extending downwardly therefrom which may include one or more external barbs 56 as shown in FIGS. 13-14. With reference to FIGS. 1-5, the stem 54 may interface with components of the water supply system 51, including a connector tube 58 and a wick 60. The stem 54 is arranged to be coupled to (e.g. inserted into) the connector tube 58, wherein the barbs 56 facilitate securing the connector tube 58 to the vaporizer housing 34, such as via an interference fit with the stem 54 received within the connector tube 58. In a non-limiting example, three barbs 56 may be provided and may be geometrically sized to fit into a 0.375 in. ID connector tube 58 to hold the connector tube 58 snugly around the stem 54 to prevent water from escaping and to prevent contaminants from getting into the water mist stream. The stem 54 includes a lumen 62 (see FIGS. 5 and 14) which is arranged to receive the wick 60. In one or more embodiments, the lumen 62 may be tapered, with a larger diameter distal end 63 for easy insertion of the wick 60, and tapering to a smaller diameter proximal end 65 closer to the ultrasonic emitter 50 to secure the wick 60 within the vaporizer housing 34.

One possible configuration of the connector tube 58 and wick 60 is depicted in FIG. 15. The connector tube 58 may be constructed from an ultra-soft PVC material, such as TYGON®, and may be sterilized prior to use. The connector tube 58 may be very flexible but yet rigid enough to prevent the wick 60 from being discharged or damaged. In one or more embodiments, the connector tube 58 is connected to a water reservoir 64 configured to contain water, such as a water bag described below with reference to FIG. 16, and the connector tube 58 protects the wick 60 and creates a sealed environment to hold water to keep the wick 60 charged for transmitting water to the ultrasonic emitter 50. The connector tube 58 can be removed from the stem 54 for replacing the wick 60, and the connector tube 58 itself may be disposable to prevent any kind of bacterial colonization.

The wick 60 is in fluid communication with the water reservoir 64 and provides safe and controllable transmission of water from the water reservoir 64 to the ultrasonic emitter 50. In one non-limiting embodiment, the wick 60 may be made from a polyester-cotton composite. By the forces of capillary action, water may be transferred from the water reservoir 64 into the wick 60 at a rate and volume dependent on the wick material absorptivity, the wick cross-sectional area, and the water exposure time. In one or more embodiments, the wick 60 may have approximately half its length within the water reservoir 64 and approximately half its length within the connector tube 58. For example, the wick 60 may be cut to approximately 18 inches in length, with approximately 9 inches extending through the connector tube 58 to the ultrasonic emitter 50 and the remaining 9 inches inside the water reservoir 64, keeping the wick charged with water to allow sustained use over time. It is understood that alternative mechanisms for transporting water to the ultrasonic emitter 50 are also contemplated.

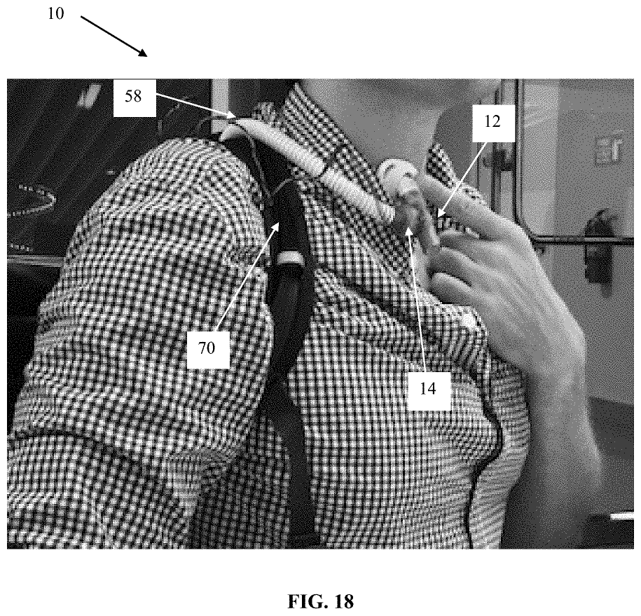

Turning to FIGS. 16-18, in one or more embodiments the portable humidification apparatus 10 may be configured as a backpack 66 or another storage container that may be easily worn or carried by the patient. The water reservoir 64, such as in the form of a refillable water bag, may be disposed in the backpack 66 and may contain a sterile water supply. The water reservoir 64 may be washable and reusable and, in one example, may have a capacity large enough to operate the apparatus 10 for 24 hours without requiring refilling. As shown in FIG. 17, a control panel or user interface 68 may be provided in electrical communication (wired or wireless) with the PCB 52, wherein the PCB 52 may be disposed in proximity to the user interface 68, such as with both the PCB 52 and the user interface 68 mounted within and supported by a remote housing 69. The remote housing 69 may be secured to the backpack 66, such as on a strap 70 thereof where it is easily accessible by a patient or medical professional during use of the apparatus 10. The user interface 68, in communication with the PCB 52, may be configured to allow a user to control such parameters as the power state (on/off), the humidity level, and/or temperature of the water mist generated by the ultrasonic emitter 50.

It is also contemplated that a heating element 72 could be disposed within the vaporizer assembly 14 downstream from the ultrasonic emitter 50 (see FIG. 5) to heat the water mist en route to the user in order to further enhance mucociliary clearance and to optimize the comfort of the user. Alternatively, the heating element 72 could heat water in the water reservoir 64 upstream from the ultrasonic emitter 50. The heating element 72 may be in electrical communication with the PCB 52 and may be powered via the power supply 55 and controlled by the microcontroller unit 53 as described above with reference to FIG. 12. The microcontroller unit 53 may control how much current will drive the heating element 72 and allow the user to control how much heat is provided via the user interface 68. Heating the water mist or water may make the breathing experience more pleasurable for the user by simulating the heating and humidifying that occurs naturally in the body.

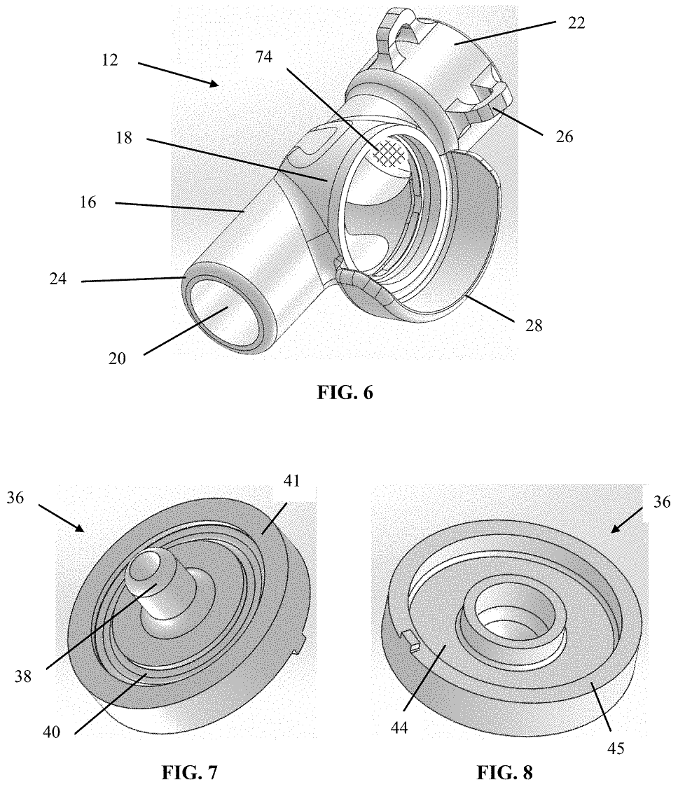

As described above, since the apparatus 10 is portable, depending on the user's environment, the likelihood of digesting airborne particulates may be high. In one or more embodiments, a particulate filter 74 (FIG. 6) may be installed within the adapter 12 in the central lumen 20 inline to the patient's breathing path, either proximal or distal to the branch portion 18, to prevent unwanted debris from being consumed by the patient. The filter 74 may have layers fine enough to block large particulates that could obstruct breathing but not so fine as to significantly reduce air flow. Due to the natural mucus development process, the filter 74 may be removable for cleaning and/or replacement.

The flexibility of the portable humidification apparatus 10 described herein lends itself to use in conjunction with other breathing devices that require or benefit from conditioning of the gas to be consumed. Currently, there are a few common ways to deliver portable consumable air to a patient, i.e. a face mask or nasal cannula. Regardless of the breathing device used, the portable humidification apparatus 10 can easily connect inline in series with the device given the appropriate adapters are attached. Furthermore, the portable humidification apparatus 10 is bidirectional which gives it the ability to be installed at either the distal or proximal end and have the water mist propel into the gas flow and mix appropriately.

The portable humidification apparatus 10 described herein will provide patients with adequate humidification of the inspired air, thereby preventing mucus from hardening in the airway, preventing airway obstruction, and improving mucociliary clearance. With the portable humidification apparatus 10 functioning to keep the airway moisturized, there is also lesser likelihood of bleeding in the airway since the airway lining will not be dry and brittle and prone to abrasions from the tracheostomy tube moving around the airway with neck motion.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A portable humidification apparatus for a patient having a tracheostomy including a tracheostomy tube, the apparatus comprising:
an adapter having a central lumen and arranged to be coupled to the tracheostomy tube, the adapter including a first magnet disposed therein;
a vaporizer assembly configured to be removably coupled to the adapter in fluid communication with the central lumen and including a second magnet disposed therein, the first magnet and the second magnet releasably connecting the adapter and the vaporizer assembly and allowing for relative rotation between the adapter and the vaporizer assembly when connected; and
an ultrasonic emitter disposed within the vaporizer assembly for generating a water mist from water in a water supply system in fluid communication with the vaporizer assembly, wherein the water mist is ducted from the vaporizer assembly to the central lumen of the adapter to provide humidification of air inspired by the patient through the tracheostomy tube.

2. The apparatus of claim 1, wherein the adapter includes a main portion having a first end and a second end, and a branch portion extending from the main portion, wherein the central lumen is disposed in the main portion and the branch portion is in fluid communication with the central lumen, wherein the first end is arranged to be coupled to the tracheostomy tube and the second end is open to an external atmosphere.

3. The apparatus of claim 2, wherein the first magnet is disposed within the branch portion.

4. The apparatus of claim 1, wherein the vaporizer assembly includes a vaporizer housing and a cover received on the vaporizer housing, wherein the cover includes a central port through which the water mist can be expelled, the central port sized to be received by the adapter to transfer the water mist into the central lumen.

5. The apparatus of claim 1, wherein the vaporizer assembly includes a seal arranged to receive the ultrasonic emitter.

6. The apparatus of claim 1, further comprising a printed circuit board (PCB) including a microcontroller unit and a power supply in electrical communication with the ultrasonic emitter.

7. The apparatus of claim 6, further comprising a user interface in electrical communication with the PCB, wherein the user interface is configured to allow selection of at least one of a power state, a humidity level, and a temperature of the water mist.

8. The apparatus of claim 6, further comprising a heating element in electrical communication with the PCB and disposed in the vaporizer assembly downstream from the ultrasonic emitter to heat the water mist generated by the ultrasonic emitter.

9. The apparatus of claim 1, further comprising a particulate filter removably disposed within the central lumen.

10. A portable humidification apparatus for a patient having a tracheostomy including a tracheostomy tube, the apparatus comprising:
an adapter including a main portion having a central lumen, the main portion having a first end arranged to be coupled to the tracheostomy tube and a second end open to an external atmosphere, and a branch portion extending from the main portion and in fluid communication with the central lumen, the adapter including a first magnet disposed in the branch portion;
a vaporizer assembly configured to be removably coupled to the adapter in fluid communication with the central lumen and including a second magnet disposed therein, the first magnet and the second magnet releasably connecting the adapter and the vaporizer assembly and allowing for relative rotation between the adapter and the vaporizer assembly when connected;
a water supply system in fluid communication with the vaporizer assembly; and
an ultrasonic emitter disposed within the vaporizer assembly for generating a water mist from water in the water supply system, wherein the water mist is ducted from the vaporizer assembly to the central lumen of the adapter to provide humidification of air inspired by the patient through the tracheostomy tube.

11. The apparatus of claim 10, wherein the water supply system includes a water reservoir configured to contain water, a connector tube coupled to the water reservoir, and a wick disposed within the connector tube and in fluid communication with the water reservoir, wherein the wick is arranged to transmit water from the water reservoir to the ultrasonic emitter.

12. The apparatus of claim 11, wherein the vaporizer assembly includes a hollow stem arranged to be coupled to the connector tube, the stem including a lumen which is arranged to receive the wick.

13. The apparatus of claim 10, wherein the vaporizer assembly includes a vaporizer housing and a cover received on the vaporizer housing, wherein the cover includes a central port through which the water mist can be expelled, the central port sized to be received by the adapter to transfer the water mist into the central lumen.

14. The apparatus of claim 10, further comprising a printed circuit board (PCB) including a microcontroller unit and a power supply in electrical communication with the ultrasonic emitter, and a user interface in electrical communication with the PCB, wherein the user interface is configured to allow selection of at least one of a power state, a humidity level, and a temperature of the water mist.

15. A portable humidification apparatus for a patient having a tracheostomy including a tracheostomy tube, the apparatus comprising:
an adapter including a main portion having a central lumen, the main portion having a first end arranged to connect to the tracheostomy tube and a second end open to an external atmosphere, and a branch portion extending from the main portion and in fluid communication with the central lumen, the adapter including a first magnet disposed therein;
a vaporizer assembly removably coupled to the adapter to be in fluid communication with the central lumen and including a second magnet disposed therein, the first magnet and the second magnet releasably connecting the adapter and the vaporizer assembly and allowing for relative rotation between the adapter and the vaporizer assembly when connected;
a water supply system in fluid communication with the vaporizer assembly, the water supply system including a water reservoir configured to contain water;
an ultrasonic emitter disposed within the vaporizer assembly for generating a water mist from water in the water reservoir, wherein the water mist is arranged to flow from the vaporizer assembly to the central lumen of the adapter to provide humidification of air inspired by the patient through the tracheostomy tube; and
a storage container arranged to be worn or carried by the patient and housing the water reservoir.

16. The apparatus of claim 15, wherein the water supply system further includes a connector tube coupled to the water reservoir, and a wick disposed within the connector tube and in fluid communication with the water reservoir, wherein the wick is arranged to transmit water from the water reservoir to the ultrasonic emitter.

17. The apparatus of claim 16, wherein the vaporizer assembly includes a hollow stem arranged to be coupled to the connector tube, the stem including a lumen which is arranged to receive the wick.

18. The apparatus of claim 15, wherein the vaporizer assembly includes a vaporizer housing and a cover received on the vaporizer housing, wherein the cover includes a central port through which the water mist can be expelled, the central port sized to be received by the adapter to transfer the water mist into the central lumen.

19. The apparatus of claim 15, further comprising a printed circuit board (PCB) including a microcontroller unit and a power supply in electrical communication with the ultrasonic emitter, and a user interface in electrical communication with the PCB, wherein the user interface is configured to allow selection of at least one of a power state, a humidity level, and a temperature of the water mist.

20. The apparatus of claim 19, wherein the storage container includes a remote housing for supporting the PCB and the user interface.
